(19) United States
(12) Patent Application Publication
(10) Pub. No.: US 2007/0123757 A1
(43) Pub. Date: May 31, 2007

(54) Neuropsychological assessment platform (NPAP) and method

(76) Inventor: Alexander B. Chervinsky, Brooklyn, NY (US)

Correspondence Address: David Aker, 23 Southern Road, Hartsdale, NY, 10530, US

(21) Appl. No.: 11/586,029

(22) Filed: Oct. 24, 2006

Related U.S. Application Data
(60) Provisional application No. 60/729,564, filed on Oct. 24, 2005.

(51) Int. Cl. A61B 5/00

(52) U.S. Cl. 600/300

(57) ABSTRACT

A computerized system designed as a professional tool to administer psychological and neuropsychological tests to human subjects using multimedia technology to present and collect audiovisual and graphomotor response data, and continuously accumulate normative and clinical data in a centralized database that is remotely accessible via the internet. A set of integrated hardware and software components utilize the internet for data transfer and access. Local testing modules administer tests to subjects with minimal examiner involvement, use audiovisual test presentation, and collect response data that includes audiovisual, graphic and touch responses. The local testing modules are remotely connected to a centralized data bank that stores and accumulates subject data, and can be remotely accessible for clinical comparison of individual subjects for diagnostic purposes, or for group analyses for research purposes. Test administration and database software are a part of the system. The system is expandable to incorporate additional local test software. Available technology is integrated to improve the testing methodology, data collection, assessment throughput, normative data availability, normative and clinical database expandability, and potential for diverse and creative data analysis.

This application claims priority from U.S. provisional application Ser. No. 60/729,564 filed on Oct. 24, 2005, which is incorporated herein by reference, in its entirety.

### BACKGROUND OF THE INVENTION

1. Field of the Invention

This invention relates to the field of Clinical Neuropsychology and Neuropsychological Assessment. Clinical Neuropsychology is an applied science concerned with the behavioral expression of brain function and dysfunction (Lezak, 1995). Neuropsychological assessment involves administration of standardized tests of various cognitive functions and emotional status to help elucidate and quantify behavioral changes that may have resulted from central nervous system disease. The present invention relates to apparatus, methods, etc. in which a computerized system administers psychological and neuropsychological tests to human subjects in a highly standardized, yet intuitive manner, requires minimal examiner involvement, accumulates data from multiple testing locations, and permits remote access to the data for clinical and research purposes.

2. Background Art

There are multiple neuropsychological assessment tools, including the prevalent traditional paper and pencil tests (Lezak, 1995; Spreen and Strauss, 1998), as well as computerized and internet-based tests (Butcher, Perry, and Hahn, 2004; Anger, 2003; Letz, 2003; Kane and Kay, 1992). Despite the breadth in variety of the developed testing tools significant limitations in testing efficiency, throughout, and data collection restrain scientific advancement.

Limitations of Traditional Instruments Prevalent in Neuropsychological Assessment

The most commonly used procedures currently implemented in neuropsychological assessment are paper and pencil tests. These share four basic limitations which significantly restrict the throughput of neuropsychological assessment procedures, including: 1) individual test administration; 2) inefficient data collection and sharing; 3) narrow amount of information obtained from test administration, and 4) variability in the way the tests are administered.

1) Low throughput due to individual test administration. Most of the currently used neuropsychological tests are designed for individual administration, making the testing procedures inefficient and time consuming. According to Sweet, Peck, Abramowitz, and Etzweiler (2002) test administration takes on the average 4.92 hours (SD=2.24) with an additional 1.2 hours needed for scoring (SD=0.78). In fact, test administration is the component of neuropsychological assessment requiring the largest amount of time. Those tests that do not require individual administration still require time for scoring. Quite a number of neuropsychologists, working in various settings use testing assistants to improve their throughput. Abovementioned authors found that among their sample of 1,352 respondents 51.2% indicated that they use testing assistants when conducting neuropsychological evaluations. Interestingly, use of testing assistants was associated with a greater number of testing hours. While use of testing assistants permits a single professional to evaluate more people, throughput is still quite limited by individual test administration.

2) Inefficient data collection, sharing, and integration. Normative and clinical data are crucial as they provide the basis of comparison for test results. The normative and clinical data available with the majority of neuropsychological instruments is limited due to inefficiencies and expense of data collection sharing, and integration.

Mitrushina, Boone and D'Elia in their Handbook of Normative Data for Neuropsychological Assessment (1999) state, “. . . although neuropsychological assessment procedures are widely available, there is a relative scarcity of normative data for most tests.” (p. 6). This scarcity relates to the expense and labor intensity of data collection which in turn relates to the low throughput of current data collection techniques. Typically, the normative data are collected prior to test publication and are supplied with the printed test manual. Additional research on the test is reported in professional publications. There is no readily available method for augmenting the original norms with the new research data.

For a number of neuropsychological tests normative data are available from different sources and populations. It falls upon a clinician to select the most appropriate normative group from the available sources, none of which may be ideal. According to Mitrushina et al. (1999), “A frequent difficulty one encounters is that use of one set of norms may suggest that the patient is performing in the impaired range while use of another normative sample may suggest that performance is within normal limits.” (p.6). Meta-analysis technique has been used to integrate information from various studies. However, such research is limited by a small number of commonly reported variables, as well as by the amount of effort involved in finding and organizing such information. There is no currently available method that allows sharing of data between studies in order to select a particular demographic or clinical group for clinical comparison.

Test performance in a population can change over time. Uttl and Van Alstine (2003) found that Vocabulary subtest scores of the Wechsler Adult Intelligence Scale (Wechsler, 1955; Wechsler, 1981; Wechsler, 1997a) have been rising over the past decades. At this time, a normal older adult (˜65 years old) with average intelligence is expected to receive a score equivalent to 124 IQ points (Superior range) on the WAIS Vocabulary test (normed between 1953 and 1954), and 112 IQ points (High Average range) on the WAIS-R (Wechsler, 1981) Vocabulary test (normed between 1976 and 1980). These findings suggest that systematic norm updates are necessary for accurate assessment of levels of functioning. However, current tools make these updates very laborious, expensive, and limited in scope.

Tests with the best normative samples provide data for over a thousand individuals with demographic characteristics comparable to that described in the recent US Census. Subdividing or stratifying the sample according to demographic characteristics such as age and education is desirable as it allows comparison of a subject's performance to that of his or her peers. Therefore, any score deviation is more likely to be due to some sort of abnormality rather than to testing/sampling artifact. While the total sample size may be impressive, stratification for even one or two demographic variables results in small representations within the individual cells. The Wechsler Memory Scale-Third Edition (WMS-III; 1997b) was co-developed with the WAIS-III, and was standardized on a sample of 1,250 adults divided into 13 age groups. The standardization sample is representative of the US in regard to gender, ethnicity, educational level, and geographic region. However, for individual normative comparison only age stratification is available. With age stratification the numbers of individuals in each of the age groups is between 75 and 100.

Clinical group comparison can help with diagnostic attribution of abnormal findings. The WAIS-III, WMS-III Technical Manual provides information on several clinical groups including Alzheimer's, Huntington's, Parkinson's Diseases, Traumatic Brain Injury, etc. However, the sample sizes for each of the disorders are small: 35, 15, 10, and 22 cases, respectively. No age stratification is provided for the clinical samples.

In order to stratify data along several parameters and have representative numbers of observations in each of the cells large numbers of individuals need to be tested. Performance of various clinical groups with different types of pathology is important to have for clinical comparison and differential diagnosis. Demographic stratification of clinical data is important for the same reasons as normative data. Systematic normative and clinical data updates are important to keep up with changes in test performance in the population. The efficiency, sharing, and integration limitations in the current testing methods do not permit such data collection.

3) Narrow amount of information obtained from test administration. The amount of information collected during test administration is rather narrow. Potentially useful information is irretrievably lost with current testing methods. Test data obtained from the traditional paper and pencil tests consists of the examiner's written record of the subject's responses, the time it took the subject to complete a particular task, the writing or drawing that the subject produced, and some behavioral observations (in the form of written notes or examiner's memory of what the subject did).

For example, the test protocol for the Rey-Osterrieth Complex Figure Test (Rey, 1941; Osterrieth, 1944) consists of the drawings made by the subject and the time it took to complete the task. Depending on the mode of administration, the drawing may be completed with different color pencils with a record of color sequence, to aid in assessment of the constructional strategy. Alternately, there may be an accompanying drawing completed by the examiner containing the sequence of the subject's construction. There may be notes on the subject's behavior during test administration.

Much of the potentially useful information such as the latency of the response, direction of gaze, style of pencil grip, time spent examining the stimulus, facial expression during task performance, representation of the actual motor activity, verbalizations, etc. are lost. No matter how conscientious the examiner, how good are his or her memory capacities and how careful he or she is at taking written notes, some of the information will be omitted. Having a more complete record of the subject's behavior during testing, especially if collected in an objective and standardized fashion will greatly enhance the clinical and research utility of psychological and neuropsychological testing. The importance of such information is uncertain. However, the irretrievable loss of this information prohibits future systematic examination of variables that may be of interest.

4) Variability in test administration. Despite efforts to standardize test administration current methods allow substantial variability in these procedures that is a potential source of error. Reliability and stability coefficients of our commonly used measures, while decent, are far from ideal even for well-standardized tests. It is considered desirable for reliability coefficients to be at 0.80 or above. The WAIS-III/WMS-III Technical manual (Wechsler, 1997c) provides tables of reliability coefficients for these tests. For WAIS-III all IQ and Index score reliabilities are 0.86 or above. However, for the subtests (14 separate tasks comprising the WAIS-III), 50 of the 182 (or 27%) of the reliability coefficients fall below 0.80 (range of low coefficients being 0.50 to 0.79). For the WMS-III the proportion of correlation coefficients below 0.80 is even higher: 18 of 104 (or 17%) Index scores, and 70 out of 143 (or 49%) subtest scores (range 0.64 to 0.79). Test-retest stability for the WAIS-III was described in the Technical Manual as “adequate” (p.57). The same descriptor is not provided for the WMS-III stabilities. For the WAIS-III subtests, 29 of 56 (or 52%) reported uncorrected stability coefficients are below 0.80, and 10 out of 56 (or 18%) are below 0.70. For the WMS-III subtests, 21 out of 22 (or 95%) of the uncorrected stability coefficients fall below 0.80, and 10 of the 22 (or 45%) fall below 0.70.

Test manuals usually focus attention on the importance of following the testing procedures with minimal deviations. Some test batteries, like the Halstead-Reitan Neuropsychological Test Battery (Reitan and Wolfson, 1985), even ask that the examiner learn the test instructions verbatim. However, there are invariable differences in which test procedures are administered. These differences may be very subtle, relating to the speed of presentation of instructions, breaks in phrasing, voice quality, or voice intonation and modulation, direction of the examiner's gaze at the materials, frequency and appropriateness of eye contact, etc. Other factors may include subtle changes in verbal instructions, familiarity and facility in the manipulation of test materials. This is aside from the procedures to “test limits” by allowing additional time for task completion or providing cues. Variation on the procedures may or may not be specifically addressed in the test administration instructions, but data are not typically provided as to the effects of such altered test administration.

Additional differences during test administration include examiner's appearance, gender, and ethno-cultural background. Even factors such as height, hair color, and attire introduce additional variation. There may be the divergent influence of interpersonal factors relating to the quality of rapport between the examiner and the subject. It is not entirely clear how these factors influence test performance individually or in combination. Information regarding the amount of error present in measurements is described in studies such as those summarized above for the WAIS-III and WMS-III. Relatively little is known about the sources of error for the majority of tests. However, it is quite possible and even likely that in assessment of subtle psychological phenomena, variations in test administration can introduce noise artifacts that may obscure or even overwhelm the phenomenon of interest. Eliminating such sources of error is likely to enhance the utility of neuropsychological procedures.

Limitations of Currently Used Computerized Tests

Computerized psychological and neuropsychological tests in current use provide partial solutions to the problems discussed above, yet still suffer many of the limitations of the traditional tests. Overall, test administration is more firmly standard, detailed timing parameters are collected, and scoring accuracy is improved. The limitations remain with regard to low throughput due to individual administration, inefficient data collection and sharing, and narrow range of collected data. Tests are developed in a form of software and there is variability in test administration that relates to the specific equipment that is used and the testing environment. Many currently available computer tests have an additional limitation related to the interface between the subject and the computer. The newer test batteries utilize some of the recent technological innovations, but none make wide-ranging use of current technology to integrate multimedia presentation, intuitive interface, collection of comprehensive audio-visual and graphomotor behavioral data, speech and pattern recognition technology, centralized [expandable] data base, and internet data access and sharing.

A number of the frequently used computerized psychological and neuropsychological tests such as the Conners' CPT-II (Conners and MHS Staff, 2002), and the computer administered Wisconsin Card Sorting Test (WCST: Heaton, Chelune, Talley, Kay, and Curtiss, 1993; Heaton and PAR Staff, 1999) use individual administration, examiner instruction or online written instructions, and keyboard entry of responses. The tests are distributed as software, results are stored on a local PC and are not centrally compiled or shared. Similar limitations are found among the specially developed neuropsychological test batteries such as the ANAM (Kabat, Kane, Jefferson and DiPino, 2001), and COGSCREEN (Kay, 1995). These tests are administered with written online instructions, keyboard or mouse entry of responses and local data storage. The range of recorded responses is limited to the key or mouse entries and timing.

Internet based tests such as the Concussion Resolution Index (CRI; Erlanger, Feldman, Kutner, Kaushik, Kroger, Festa, Barth, Freeman, and Broshek, 2003) is administered with limited examiner involvement and allows remote access for testing and data analysis. While this type of testing permits centralized data accumulation, the system is limited by the interface with written instructions, keyboard responses, no audiovisual recording or drawing responses.

Several computer-administered test batteries partially utilize recent technological innovations. The Cambridge Neuropsychological Test Automated Battery (CANTAB; Robins, James, Owen, Sahakian, McInnes, and Rabbitt, 1994; Robins, James, Owen, Sahakian, Lawrence, McInnes, and Rabbitt, 1998) utilizes a touch screen monitor for data acquisition. However, the test is individually administered with instructions provided verbally by an examiner who remains with the subject through the testing session. The amount of behavioral information recorded is narrow and there is no central compilation of the data.

Three test batteries use recorded voice instruction, minimal examiner involvement, and conditional responsiveness to subject's keyboard strokes (Aharonson and Korczyn, 2004; BARS; Rohlman, Gimenes, Eckerman, Kang, Farahat, and Anger, 2003; and NES3: Letz, Green, and Woodard, 1996; Letz, GiIorio, Shafer, Yeager, Schomer, and Henry, 2003). None of these batteries collect audiovisual data, use speech recognition technology, or allow central database compilation, provide an expandable database or remote access to the results.

### SUMMARY OF THE INVENTION

This invention, the Neuropsychological Assessment Platform (NPAP), is a computerized system designed as a professional tool to administer psychological and neuropsychological tests to human subjects, collect and accumulate data from various locations and to make that data widely available for clinical and research purposes. NPAP contains integrated hardware and software components and utilizes the internet for data transfer and access. The system includes local testing modules that administer tests to subjects with minimal examiner involvement, use audiovisual test presentation, collect response data that includes audiovisual, graphic and touch responses. The local testing modules are remotely connected to a centralized data bank that stores and accumulates subject data, and can be remotely accessible for clinical comparison of individual subjects for diagnostic purposes, or for group analyses for research purposes. The system includes test and database software and is designed to permit addition of new test software and software updates. This computerized testing system integrates the latest currently available technology to improve the testing methodology, data collection, assessment throughput, normative data availability, normative and clinical database expandability, and potential for diverse and creative data analysis.

The invention is also directed to a method of performing such testing and/or assessment.

It is an object of the invention to provide a superior tool for efficient and accurate neuropsychological data collection, analysis and distribution by integration of the latest technological advances such as multimedia presentation, touch screen, audiovisual recording and internet connectivity.

It is a further object of the invention to provide for administration of a battery of neuropsychological tests covering the important domains including attention, memory, language, visuospatial/constructional, executive, and emotional/personality functions, with tests designed to minimize cultural bias.

It is another object of the invention to provide administration of tests using a multimedia audiovisual format with minimal examiner involvement.

It is another object of the invention to provide collection of comprehensive behavioral response data that will include video, audio, drawing, eye gaze, and pointing responses.

It is another object of the invention to provide an automated reaction to the subject's responses with instructions and cues.

It is another object of the invention to permit testing of subjects with no computer experience, typing ability, limited education, and cognitive difficulties.

It is another object of the invention to permit testing of several individuals simultaneously.

It is another object of the invention to provide an avenue for addition of new tests, test updates, and updates of data analysis software.

It is another object of the invention to provide for accumulation of data in a centralized location that will be remotely accessible via the internet.

It is another object of the invention to provide a system comprising the locally connected subject's station and the examiner's station, as well as the remotely accessible central data bank.

These objects and others are achieved in accordance with the invention by integrating the available electronic and data transmission components with original software into a versatile and expandable system that functions as a professional tool.

### BRIEF DESCRIPTION OF THE DRAWINGS

The foregoing aspects and other features of the present invention are explained in the following description, taken in connection with the accompanying drawings, wherein:

FIG. 1 is a block diagram of the NPAP apparatus components and connectivity.

FIG. 2 is a diagram of the components of the Subject's Station of FIG. 1 from the Subject's perspective.

FIG. 3 is a plan view diagram of the components of the Subject's Station depicted in FIG. 1 and FIG. 2. if viewed from above.

FIG. 3A is a side view of portions of the arrangement of FIG. 3.

FIG. 4 is a diagram of the components of the Examiner's Station depicted in FIG. 1 from the examiner's view.

### DETAILED DESCRIPTION OF THE PREFERRED EMBODIMENT

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

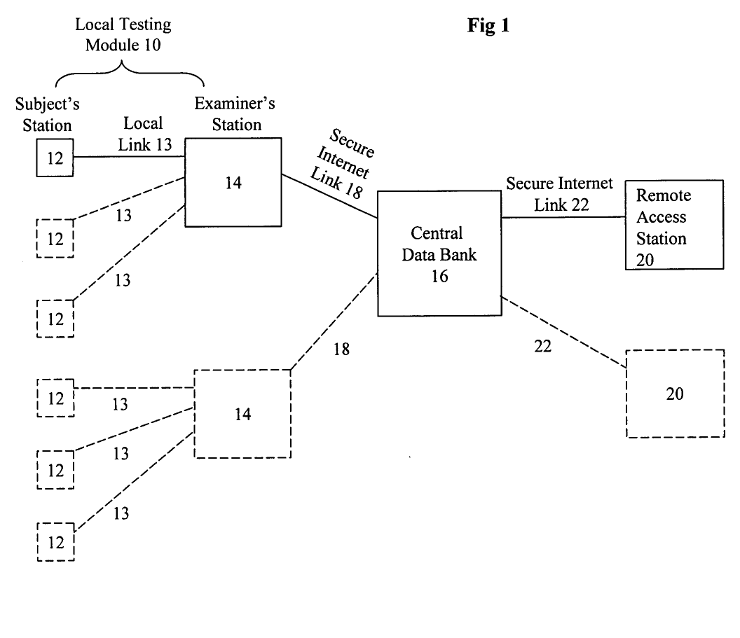

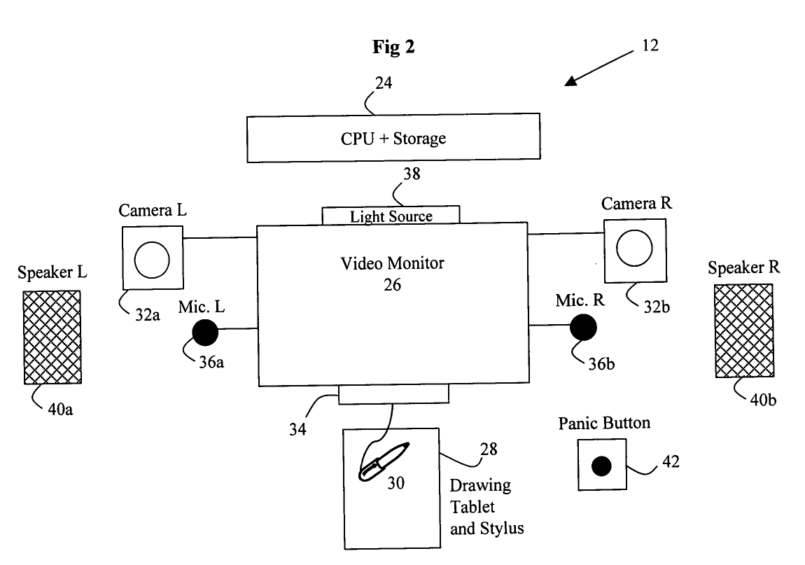

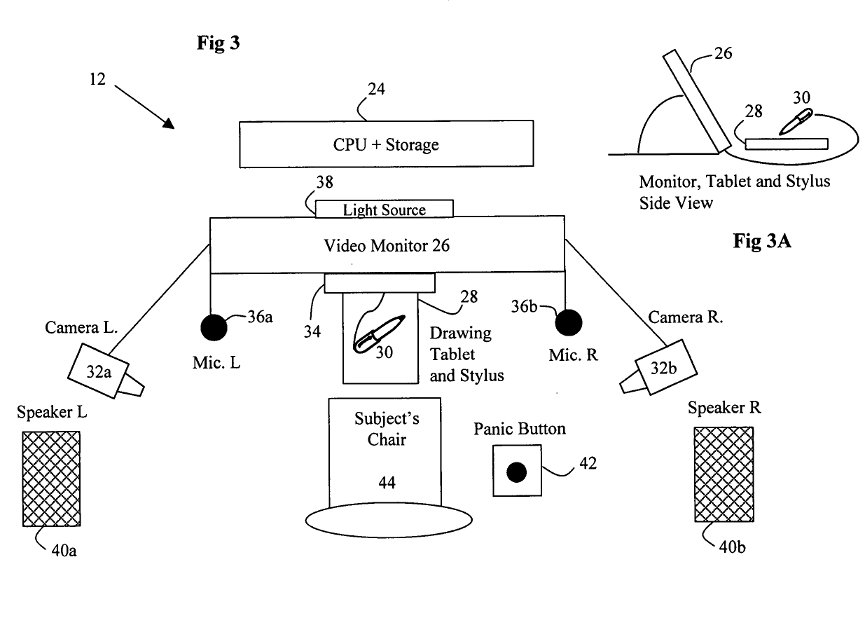

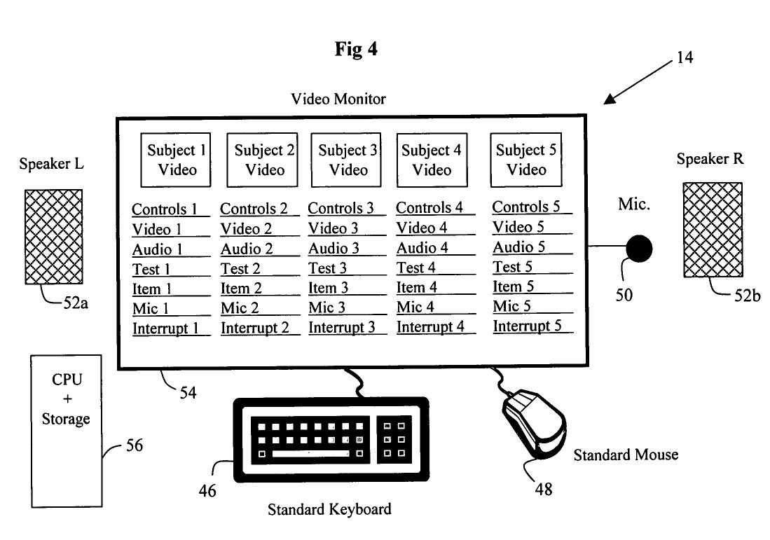

Referring to FIG. 1, FIG. 2, FIG. 3, and FIG. 4 there is shown a diagrammatic view of the NPAP apparatus incorporating features of the present invention. Although the present invention will be described with reference to the single embodiment shown in the drawings, it should be understood that the present invention can be embodied in many alternate forms of embodiments. In addition, any suitable size, shape or type of elements or materials could be used.

FIG. 1 is a block diagram of the apparatus components and connectivity. The Local Testing Module 10 represents the portion of the computerized apparatus that is located at a testing facility where subjects are physically present and examined. The Local Testing Module consists of one or more Subject's Stations 12 connected by a local network to an Examiner's Station 14. The Subject's Station 12 is equipped to present the tests and to record data. The Examiner's Station 14 controls one or more Subject's Stations 12 and is equipped to start, stop, and monitor testing. Once testing is completed the data from the Local Testing Module 10 is forwarded to a Central Data Bank 16 via a remote secure internet connection 18. The Central Data Bank 16 can receive input from multiple Local Testing Modules 10. The Central Data Bank 16 performs two principal activities. First, it accumulates and permanently stores test data. Second, it provides an interface to perform queries on the stored data. The Central Data Bank 16 is accessible by multiple Remote Access Stations 20 via secure remote internet connections 22 for examination and analysis of the stored data.

FIG. 2 is a diagram of the components of the Subject's Station 12 of FIG. 1 from the Subject's perspective. The Subject's Station 12 is designed around a custom multimedia computer 24 that is used to administer the tests to the subject. Various components illustrated in FIG. 2 are connected to computer 24 by wire or wireless links (not shown) in a manner well known in the art. The Subject's Station 12 contains a high resolution touch screen 26 allowing simple pointing responses without the need for a keyboard. An additional tablet monitor 28, having a stylus 30 is included to allow drawing or written responses. Responses by touch or stylus will also be archived. Two pan/tilt controllable camera units 32a and 32b are a part of the station, providing video of the subject, with the capability of tracking certain features on the subject. Additionally, an eye-tracking system 34, as offered by or similar to the Eyegaze Analysis System, manufactured by LC Technologies, Inc. of Fairfax, Va., U.S.A. is a part of the station to follow gaze response and relate it to the test responses. Microphones 36a and 36b are used to record verbal responses. A light source 38 provides standard illumination for video recording. An audio system, implemented by using computer 24 and having speakers 40a and 40b, provides high quality auditory stimuli. A Panic Button 42 is included to allow the subject to call the examiner for assistance. All measured inputs are globally timed and synchronized. In this way, an accurate record of the subject's total response during the test can be archived and the session recreated. All of the responses and sensory input are automatically sent to the Central Data Bank 16 via the examiner's station 14 to create the global database of test responses.

FIG. 3 is a diagram of the components of the Subject's Station depicted in FIG. 1 and FIG. 2, if viewed from above.

In FIG. 3A, the touch screen monitor is depicted in a tilted position to enhance touch responses. A subject's chair 44 indicates the subject's position relative to the system components.

FIG. 4 is a diagram of the components of the Examiner's Station 14 depicted in FIG. 1 from the examiner's view. Station 14 is designed around a computer 56. Various components illustrated in FIG. 4 are connected to computer 56 by wire or wireless links (not shown) in a manner well known in the art. The components include a conventional keyboard 46 and mouse 48, a microphone 50 and speakers 52a and 52b. With assistance from observations made on video monitor 54, station 14 is used to control multiple subject's stations 12.

By using multiplexing, a single examiner can monitor a number of subject consoles. Station 14 has the capability of multiple video and audio feeds from each of the Subject's stations 12, allowing the examiner to monitor a subject individually. Examiner's Station 14 has the capability of remote intervention during the test. Monitor 54 displays the test administration information from each of the locally connected Subject's Stations 14.

FIG. 4 shows the display for five Subject's Stations 12. The test administration information includes video input from each of the Subject's Stations 12, the test and item of the testing procedure that is being administered, as well as display of controls. The controls allow the examiner to select audio input from one of the Subject's Stations, turn on the microphone to speak with the subject, and to start or interrupt the testing procedures. All tests are downloaded from the examiner console and the full suite of data provided during the test is sent to it from the Subject's Station 12. The Examiner's Station 14 then sends the relevant data to a server in Central Data Bank 16 for inclusion in the database therein. Software may leverage existing web-based browser technology with the custom designs of the station. Using internet protocols, multiple video and audio feeds can be multiplexed into the Central Data Bank 16 from the testing stations.

Software is utilized to permit interaction between the system and the subject, as well as to assist in analysis of the test findings. The system provides auditory instructions to the subject and can automatically respond to certain subject's behaviors. For example, speech recognition software may be used to recognize and digitize simple responses (numerals, letters, [and] limited word vocabulary). The system may also have a provision to flag verbal responses that cannot be recognized for later manual correction. A limited version of automated video segmentation (Liu and Kender, 2003) may be used to allow the video to be compressed temporally into relevant segments associated with responses.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

### REFERENCES

1. Ahraronson, V., & Korczyn, A. D. (2004). Human-computer interaction in the administration and analysis of neuropsychological tests. Computer Methods and Programs in Biomedicine, 73, 43-53.

2. Allen, P., Miller, A. T., Oh, P., & Leibowitz B. (1999). Integration of Vision, Force and Tactile Sensing for Grasping. Int. Journal of Intelligent Mechatronics, 4, 129-149.

3. Allen, P., Stamos, I., Gueorguiev, A., Gold, A., & Blaer, P. (2001). AVENUE: Automated Site Modeling in Urban Environments. Third International Conference on 3D Digital Imaging and Modeling, May 28-June 1, Quebec City, 357-364.

4. Allen, P., Stamos, I., Troccoli, A., Smith, B., Leordeanu, M., & Murray, S. (2003). New Methods for Digital Modeling of Historic Sites. IEEE Computer Graphics and Applications, Nov/Dec, 32-41.

5. Anger, W. K. (2003). Neurobehavioral tests and systems to assess neurotoxic exposures in the workplace and community. Occupational and Environmental Medicine, 60, 531-538.

6. Army Individual Test Battery. (1944). Manual of Directions and Scoring. Washington, DC: War Department, Adjutant General's Office.

7. Benton, A. L., Hamsher, K. deS., Varney, N. R., & Spreen, O. (1983). Contributions to neuropsychological assessment: A clinical manual. New York: Oxford University Press.

8. Benton, A. L., & Hamsher, K. deS. (1976). Multilingual Aphasia Examination. Iowa City: Department of Neurology, University of Iowa Hospitals.

9. Benton, A. L., Hamsher, K. deS., & Sivan, A. B. (1994). Multilingual Aphasia Examination: Manual of Instructions (3rd ed.). Iowa City: AJA Associates, Inc.

10. Brown, J. (1958). Some tests of the decay theory of immediate memory. Quarterly Journal of Experimental Psychology, 10, 12-21.

11. Butcher, J. N, Perry, J., & Hahn, J. (2004). Computers in clinical assessment: historical developments, present status, and future challenges. Journal of Clinical Psychology, 60, 331-345.

12. Chervinsky, A. B., Mitrushina, M., Satz, P. (1992). Comparison of four methods of scoring the Rey-Osterrieth Complex Figure Drawing Test on four age groups of normal elderly. Brain Dysfunction, 5, 267-287.

13. Conners, C. K., & M H S Staff. (2002). Conners' Continuous Performance Test II (CPT II) [Computer Software]. North Tonawanda, N.Y.: Multi-Health Systems, Inc. (MHS)

14. Erlanger, D., Feldman, D., Kutner, K., Kaushik, T., Kroger, H., Festa, J., Barth, J., Freeman, J., & Broshek, D. (2003). Development and validation of a web-based neuropsychological test protocol for sport-related return-to-play decision-making. Archives of Clinical Neuropsychology, 18, 293-316.

15. Heaton, R. K., Chelune, G. J., Talley, J. L., Kay, G. G., and Curtiss, G. (1993). Wisconsin Card Sorting Test Manual Revised and Expanded. Odessa, FL: Psychological Assessment Resources, Inc. (PAR).

16. Heaton, R. K., and P A R Staff. (1999) Wisconsin Card Sorting Test: Computer Version 3 for Windows, Research Edition (WCST:CV3) [Computer Software]. Odessa, Fla.: Psychological Assessment Resources, Inc. (PAR).

17. Kabat, M. H., Kane, R. L., Jefferson, S., DiPino, R. K. (2001). Construct validity of selected Automated Neuropsychological Assessment Metrics (ANAM) battery measures. Clinical Neuropsychologist, 15, 498-507.

18. Kane, R. L., Kay, G. G. (1992). Computerized assessment in neuropsychology: a review of test and test batteries. Neuropsychology Review, 3, 1-117.

19. Kay, G. G. (1995). COGSCREEN: Professional Manual. Odessa, Fla.: Psychological Assessment Resources, Inc. (PAR).

20. Letz, R. (2003). Continuing challenges for computer-based neuropsychological tests. NeuroToxicology, 24, 479-489.

21. Letz, R., DiIorio, C. K., Shafer, P. O., Yager, K. A., Schomer, D. L., Henry, T. R. (2003). Further standardization of some NES3 tests. Neurotoxicology, 24, 491-501.

22. Letz, R., Green, R. C., & Woodard, J. L. (1996). Development of a computer-based battery designed to screen adults for neuropsychological impairment. Neurotoxicology and Teratology, 18, 365-370.

23. Lezak, M. D. (1995). Neuropsychological assessment (3rd ed.). New York: Oxford University Press.

24. Liu, Tiecheng, & Kender, J. R. (2003). Spatial-temporal Semantic Grouping of Instructional Video. Content, in Proceedings of International Conference on Content-based Image and Video Retrieval (CIVR).

25. Maj, M., D'Elia, L., Satz, P., Jansses, R., Zaudig, M., Uchiyama, C., Starace, F., Galderisi, S., & Chervinsky, A. B. (1993). Evaluation of two new neuropsychological tests designed to minimize cultural bias in the assessment of HIV-1 seropositive persons: A WHO study. Archives of Clinical Neuropsychology, 8, 123-135.

26. Miller, A., Allen, P., Fowler, D. (2004). In-Vivo Stereoscopic Imaging System with 5 Degrees-of-Freedom for Minimal Access Surgery. Medicine Meets Virtual Reality Conference (MMVR), Jan. 16, 2004, Newport Beach.

27. Mitrushina, M. N., Boone, K. B., D'Elia, L. F. (1999). Handbook of normative data for neuropsychological assessment. New York: Oxford University Press.

28. Oh, P., Allen, P. K. (2001). Visual Servoing by Partitioning Degrees-of-Freedom. IEEE Trans. on Robotics and Automation, 17, 1-17.

29. Osterrieth, P. A. (1944). Le test de copie d'une figure complexe. Archives de Psychologie, 30, 206-356.

30. Peterson, L. R., & Peterson, M. J. (1959). Short-term retention of individual verbal items. Journal of Experimental Psychology, 58, 193-198.

31. Robins, T. W., James, M., Owen, A. M., Sahakian, B. J., Lawrence, A. D., McInnes, L., & Rabbitt, P. M. (1998). A study of performance on tests from the CANTAB battery sensitive to frontal lobe dysfunction in a large sample of normal volunteers: Implications for theories of executive functioning and cognitive aging. Journal of the International Neuropsychological Society, 4, 474-490.

32. Robins, T. W., James, M., Owen, A. M., Sahakian, B. J., McInnes, L., & Rabbitt, P. M. (1994). Cambridge Neuropsychological Test Automated Battery (CANTAB): a factor analytic study of a large sample of normal elderly volunteers. Dementia, 5, 266-281.

33. Rohlman D. S., Gimenes L., Eckerman D. A., Kang S-K., Farahat F. M., Anger W. K. (2003). Development of the Behavioral Assessment and Research System (BARS) to Detect and Characterize Neurotoxicity in Humans. NeuroToxicology, 24, 523-531.

34. Stuss, D. T., Stethem, L. L., & Poirier, C. A. (1987). Comparison of three tests of attention and rapid information processing across six age groups. The Clinical Neuropsychologist, 1, 139-152.

35. Spreen, O., & Strauss, E. (1998). A compendium of neuropsychological tests: administration, norms, and commentary. New York: Oxford University Press.

36. Sweet, J. J., Peck, E. A., Abramowitz, C., & Etzweiler, S. (2002). National Academy of Neuropsychology/Division 40 of the American Psychological Association practice survey of clinical neuropsychology in the United States, Part I: practitioner and practice characteristics, professional activities, and time requirements. The Clinical Neuropsychologist, 16, 109-127.

37. Reitan, R. M., & Wolfson, D. (1985). The Halstead-Reitan Neuropsychological Test Battery: Theory and Clinical Interpretation. Tucson, AZ: Neuropsychology Press.

38. Rey, A. (1941). L'examen psychologique dans les cas d'encepahlopathie traumatique. Archives de Psychologie, 28, 286-340.

39. Uttl, B., & Van Alstine, C. L. (2003). Rising verbal intelligence scores: implications for research and clinical practice. Psychology and Aging, 18, 616-621.

40. Wechsler, D. (1939). Wechsler-Bellvue Intelligence Scale. New York: The Psychological Corporation.

41. Wechsler, D. (1955). Wechsler Adult Intelligence Scale. New York: The Psychological Corporation.

42. Wechsler, D. (1981). Wechsler Adult Intelligence Scale—Revised. San Antonio, Tex.: The Psychological Corporation.

43. Wechsler, D. (1997a). Wechsler Adult Intelligence Scale—Third Edition. San Antonio, Tex.: The Psychological Corporation.

44. Wechsler, D. (1997b). Wechsler Memory Scale—Third Edition. San Antonio, Tex.: The Psychological Corporation.

45. Wechsler, D. (1997c). Wechsler Adult Intelligence Scale—Third Edition; Wechsler Memory Scale—Third Edition: Technical Manual. San Antonio, Tex.: The Psychological Corporation.

46. Yoshimi, B. H., Allen, P. K. (1995). Active Uncalibrated Visual Servoing. IEEE Transactions on Robotics and Automation, 11, 516-521.

What is claimed is:

1. A system for at least one of behavioral assessment, psychological testing and/or assessment, and neuropsychological testing and/or assessment comprising: at least one Local Testing Module, said Local Testing Module comprising at least one computerized subject's testing station and at least one computerized examiner's monitoring station; said Subject's Station comprised of a plurality of input/output devices, said plurality of the input/output devices comprising: at least one visual recording device, at least one audio recording device, at least one graphomotor recording device, at least one touch-screen display device and at least one panic-communication button to communicate with a person administering and monitoring a testing procedure at the Examiner's Station; said system for psychological testing further comprising a plurality of psychological tests in form of computer software accessible by a subject of psychological testing and by the person administering and monitoring a testing procedure by using said plurality of input/output devices; said system further comprising facilities for compiling and storing, in a Centralized Data Bank, a comprehensive behavioral data input received from said plurality of the input/output devices.

2. The system for testing and/or assessment, as claimed in claim 1 further comprising a psychological testing evaluation software; said psychological testing evaluation software providing test instructions, giving cues and prompts on the basis of subject's behaviors, selecting and analyzing a compiled data, doing normative comparison and providing a result, as directed by the person performing the assessment.

3. The system for testing and/or assessment, as claimed in claim 1, further comprising two or more said local testing modules disposed in multiple remote locations and being connected to said Central Data Bank by a high speed secure internet connection.

4. The system for testing and/or assessment, as claimed in claim 2, further comprising security protocols providing authorized access to said central data bank and said Local Testing Modules.

5. The system for testing and/or assessment, as claimed in claim 1, having facilities for allowing access to all aspects of the data that is stored or compiled to be replayed at a later time by said Examiner's Station

6. The system for testing and/or assessment, as claimed in claim 1, wherein the Local Testing Module has two or more said Subject's Stations to allow testing of more than one subject.

7. The system for testing and/or assessment, as claimed in claim 1, wherein said visual recording device comprises a plurality of video cameras and software allowing analysis of direction of visual gaze.

8. The system for testing and/or assessment, as claimed in claim 1, wherein said audio recording input/output device is a plurality of microphones and a plurality of speakers.

9. The system for testing and/or assessment, as claimed in claim 1, wherein the graphomotor input/output device is a tablet and a pen stylus for graphomotor response.

10. The system for testing and/or assessment, as claimed in claim 1, wherein said Examiner's Station controls and monitors said computerized testing station.

11. The system for testing and/or assessment, as claimed in claim 9, wherein said at least one Examiner's Station starts and stops the testing and provides audiovisual monitoring of said computerized testing station.

12. The system for testing and/or assessment, as claimed in claim 1, wherein said Central Data Bank is configured to continually accept and store new data.

13. The system for testing and/or assessment, as claimed in claim 1, wherein said Central Data Bank is configured for review and analysis of recorded individual and group data.

14. The system for testing and/or assessment, as claimed in claim 1, configured for access to said Central Data Bank by a high speed secured internet connection to an authorized person performing assessment using another computer in remote location.

15. The system for testing and/or assessment, as claimed in claim 1, wherein the Central Data Bank is configured to allow authorized access to all aspects of compiled data by using another computer in a remote location.

16. The system for testing and/or assessment, as claimed in claim 1, wherein the Central Data Bank is configured to allow authorization to different levels of access.

17. A method for at least one of behavioral assessment, psychological testing and/or assessment, and neuropsychological testing and/or assessment comprising:
placing a subject to be tested at a Local Testing Module,
testing the subject in accordance with a plurality of psychological tests in a form of computer software accessible by the subject from a computer,
recording at least one of visual, audio, graphomotor, at and touch-screen response of the subject;
storing, in a Centralized Data Bank, comprehensive behavioral data input generated by the subject during testing; and
monitoring and controlling the testing from a computerized examiner's monitoring station.

18. The method of claim 17, further comprising providing a panic button usable by the subject to alert an examiner of the subject that the subject is in need of assistance.

19. The method of claim 17, further comprising using the examiner's monitoring station to monitor and control a plurality of Local Testing Modules.

20. The method of claim 17, further comprising administering and monitoring a testing procedure by using a plurality of input/output devices at said examiner's monitoring station.

21. The method of claim 17, further comprising retrieving and analyzing data from said Centralized Data Bank.

22. The method of claim 21, wherein said analyzing comprises determining norms from said data.
